(19) United States
(12) Patent Application Publication
(10) Pub. No.: US 2024/0164668 A1
(43) Pub. Date: May 23, 2024

(54) METHODS AND DEVICES FOR DETECTING INTENSITY OF LIGHT WITH TRANSLUCENT DETECTOR

(71) Applicant: Cercacor Laboratories, Inc., Irvine, CA (US)

(72) Inventors: Kevin Pauley, Lake Forest, CA (US); Cristiano Dalvi, Lake Forest, CA (US); Hung Vo, Fountain Valley, CA (US); Jesse Chen, Foothill Ranch, CA (US); Ferdyan Lesmana, Irvine, CA (US); Jeroen Poeze, Rancho Santa Margarita, CA (US); Sean Merritt, Lake Forest, CA (US)

(21) Appl. No.: 18/520,380

(22) Filed: Nov. 27, 2023

Related U.S. Application Data
(63) Continuation of application No. 16/985,497, filed on Aug. 5, 2020, now Pat. No. 11,864,890.
Application No. 16/985,497 is a continuation of application No. 15/850,755, filed on Dec. 21, 2017, now Pat. No. 10,750,984.
(60) Provisional application No. 62/437,996, filed on Dec. 22, 2016.

(51) Int. Cl. A61B 5/1455; A61B 5/00; A61B 5/145; G01J 1/16; G01N 21/31; G01N 21/47; G01N 21/49; G01N 21/59

(52) U.S. Cl. CPC A61B 5/14552; A61B 5/14532; A61B 5/14535; A61B 5/14539; A61B 5/14542; A61B 5/14546; A61B 5/4875; G01J 1/1626; G01N 21/31; G01N 21/4738; G01N 21/49; G01N 21/59; A61B 2562/0238; G01J 2001/1636; G01J 2001/1652; G01N 2021/3181

(57) ABSTRACT

An optical measurement device includes a light source, a first detector, and a second detector. The light source emits light to a measurement site of a patient and one or more detectors detect the light from the light source. At least a portion of a detector is translucent and the light passes through the translucent portion prior to reaching the measurement site. A detector receives the light after attenuation and/or reflection or refraction by the measurement site. A processor determines a light intensity of the light source, a light intensity through a tissue site, or a light intensity of reflected or refracted light based on light detected by the one or more detectors. The processor can estimate a concentration of an analyte at the measurement site or an absorption or reflection at the measurement site.

### CROSS-REFERENCE TO RELATED APPLICATIONS

The present application is a continuation of U.S. application Ser. No. 16/985,497, entitled “METHODS AND DEVICES FOR DETECTING INTENSITY OF LIGHT WITH TRANSLUCENT DETECTOR,” filed Aug. 5, 2020, which is itself a continuation of U.S. application Ser. No. 15/850,755, entitled “METHODS AND DEVICES FOR DETECTING INTENSITY OF LIGHT WITH TRANSLUCENT DETECTOR,” filed Dec. 21, 2017 and now issued as U.S. Pat. No. 10,750,984, which claims priority benefit to U.S. Provisional Application No. 62/437,996, entitled “METHODS AND DEVICES FOR DETECTING INTENSITY OF LIGHT WITH TRANSLUCENT DETECTOR,” filed Dec. 22, 2016, each of which is hereby incorporated herein by reference in its entirety.

### FIELD OF THE DISCLOSURE

The present disclosure relates to the field of non-invasive optical based sensors that include a translucent detector to detect an intensity of incident light, an intensity of light transmitted through a measurement site, and/or an intensity of light reflected or refracted by a measurement site.

### BACKGROUND

Oximetry is the measurement of the oxygen status of blood. Early detection of low blood oxygen is critical in the medical field, for example in critical care and surgical applications, because an insufficient supply of oxygen can result in brain damage and death in a matter of minutes. Pulse oximetry is a widely accepted non-invasive procedure for measuring the oxygen saturation level of arterial blood, an indicator of oxygen supply. A pulse oximetry system consists of a sensor attached to a patient, a monitor, and a cable connecting the sensor and monitor. Conventionally, pulse oximetry sensors include a light source(s) characterized by a plurality of wavelengths of known spectra transmitting optical radiation into or reflecting off a measurement site, such as, body tissue carrying pulsing blood and/or interstitial fluid. After attenuation by tissue and fluids of the measurement site, a photodetection device(s) detects the attenuated light and outputs a detector signal(s) responsive to the detected attenuated light. A signal processing device(s) process the detector(s) signal(s) and outputs a measurement indicative of a blood constituent of interest, such as glucose, oxygen saturation, methemoglobin, carboxyhemoglobin, glycated hemoglobin, respiration rate, pulse rate, total hemoglobin, other physiological parameters, or other data or combinations of data useful in determining a state or trend of wellness of a patient.

The sensor is typically attached to a patient's finger or toc, or a very young patient's foot. For a finger, the sensor is configured so that the light source (for example, an LED) projects light through the fingernail and into the blood vessels and capillaries underneath. The photodiode is positioned at the fingertip opposite the fingernail so as to detect the transmitted light as it emerges from the finger tissues.

### SUMMARY

The present disclosure provides an improved sensor design which enables more accurate estimates of incident light, as well as a concentration of one or more analytes of interest present at a measurement site. An optical measurement device according to the present disclosure can include a light source, a first detector, and a second detector. The light source can be configured to emit light to a measurement site, and the first detector can be configured to receive incident light from the light source. At least a portion of the first detector can be translucent and the incident light can pass through the translucent portion prior to reaching the measurement site. The second detector can be configured to receive the light after attenuation by the measurement site.

The device of the preceding paragraph may also include any combination of the following features described in this paragraph, among others described herein. The optical measurement device can be a pulse oximetry device. The measurement site can include a tissue site of a patient. The measurement site can include biological material. The measurement site can include non-biological material.

The device of any of the preceding paragraphs may also include any combination of the following features described in this paragraph, among others described herein. The first detector can be proximal to the light source with respect to the measurement site. The first detector can be proximal to the light source with respect to the second detector. The second detector can be distal to the first detector with respect to the measurement site.

The device of any of the preceding paragraphs may also include any combination of the following features described in this paragraph, among others described herein. The light source can include one or more light emitting diodes (LEDs). The one or more LEDs can be superluminescent light emitting diode (SLED). The light emitted by the light source can be optical radiation of red, infrared, near infrared or other wavelengths. The first detector can be configured to detect the incident light in real-time. The device can include a housing configured to house the light source. The device can further include flex circuitry, flexible connections, or flexible cabling configured to electrically connect the first detector to the housing. The second detector can include one or more multi-detectors, such as a set of multi-detectors.

The device of any of the preceding paragraphs may also include any combination of the following features described in this paragraph, among others described herein. The first detector can include an antireflective (AR) coating layer, a photodiode layer, and a wafer layer. The photodiode layer can include an Indium-Gallium-Arsenide (InGaAs) PIN photodiode and/or a silicon PIN photodiode. The photodiode layer can be as thin as 2 nanometers. The wafer layer can include an N-type Indium Phosphide (N—InP) wafer. The wafer layer can be about 11 micrometers thick. The first detector further can include a metalized surface for anode/cathode bond pads. The first detector can absorb less than 5% of the light emitted by the light source. One or more physiological parameters can be determined based at least in part on a detected light intensity of the light source by the first detector and the detected light intensity after absorption by the tissue site by the second detector.

The present disclosure also provides a method of estimating a concentration of an analyte of interest at a measurement site. A monitoring system can attach to, attached near, or be directed at the measurement site. The method can include receiving incident light from a light source at a first detector. At least one portion of the first detector can be translucent and the incident light can be received by and pass through the at least one translucent portion before reaching a measurement site. The method can further include determining an intensity of incident light based at least in part on the incident light received by the first detector. The method can further include receiving attenuated light at a second detector. The attenuated light can be the light which emerges from the measurement site. The method can further include determining intensity of light through the measurement site based at least in part on the attenuated light received by the second detector. The method can further include estimating a concentration of an analyte of interest at the measurement site based at least in part on the intensity of the incident light and the intensity of the attenuated light.

The method of the preceding paragraph may also include any combination of the following features described in this paragraph, among others described herein. The measurement site can include a tissue site of a patient. The measurement site can include biological material. The measurement site can include non-biological material. The method can further include estimating one or more physiological parameters based at least in part on the estimated concentration of the analyte of interest at the measurement site. The method can further include predicting blood glucose concentration.

The present disclosure also provides an optical measurement device that includes a light source configured to emit light to a measurement site, and a detector that includes a translucent portion. The detector can be configured to detect light as it passes through that translucent portion.

The device of the preceding paragraph may also include any combination of the following features described in this paragraph, among others described herein. The optical measurement device can be a pulse oximetry device. The measurement site can include a tissue site of a patient. The measurement site can include biological material. The measurement site can include non-biological material. The detector can be configured to detect incident light of the light source before light from the light source is attenuated by the measurement site. The detector can be configured to detect light reflected or refracted by the measurement site.

The device of any of the preceding two paragraphs may also include any combination of the following features described in this paragraph, among others described herein. The detector can be a first detector and can be configured to detect light before the light is attenuated by the measurement site. The device can further include a second detector configured to receive the light after attenuation by the measurement site. The first detector can be proximal the tissue site with respect to the second detector.

A system, method, or device having any of the features described herein or illustrated in the drawings.

This disclosure describes embodiments of non-invasive methods, devices, and systems for measuring a blood and/or interstitial fluid constituent (which may also be referred to as an analyte), and/or substance such as oxygen, carboxyhemoglobin, methemoglobin, total hemoglobin, glycated hemoglobin, glucose, proteins, lipids, a percentage thereof (for example, saturation), or for measuring many other physiologically relevant patient characteristics. These characteristics can relate, for example, to pulse rate, hydration, trending information, blood oxygen level and analysis, and the like. For example, these measurements can be taken using a non-invasive optical sensor.

This disclosure describes embodiments of non-invasive methods, devices, and systems for detecting intensity of light emitted from a light source of an optical measurement device. The optical measurement device can include a pulse oximeter and/or a spectrophotometer. An optical measurement device or system can determine the power going into a patient's tissue and estimate a concentration of an analyte of interest at the tissue site. In some cases, the optical measurement device or system can estimate absorption of the tissue.

The optical measurement device can be configured to measure tissue of a patient and includes a light source and one or more detectors. The optical measurement device can be attached to the patient measurement site using adhesives, straps, clips, bandages or other attachment devices. The light source can project light through a tissue site of a patient. A detector can detect the light as it emerges from the light source. At least a portion of the detector can be translucent. Light projected from the light source can pass through the translucent portion of the detector prior to reaching the tissue site of the patient. A detector can also detect light as it emerges from the patient's tissue. In addition or alternatively, a detector can also detect light that is reflected or refracted from the patient's tissue. In some cases, these are the same sensor. In some cases, these are different sensors.

The present disclosure also provides that one or more physiological parameters can be determined based at least in part on a detected light intensity of a light source by a first detector and a detected light intensity after absorption by a tissue site by a second detector. The second detector can include a set of detectors.

The present disclosure also provides embodiments for a method of estimating a concentration of an analyte of interest at a tissue site of a patient by a patient monitoring system is provided. The method includes receiving light from a light source at a first detector wherein at least one portion of the first detector is translucent and the light is received by and passes through the at least one translucent portion before reaching a tissue site of a patient; determining a light intensity of the light source based at least in part on the light received by the first detector; receiving attenuated light at a second detector, wherein the attenuated light is the light which emerges from a tissue site of the patient; determining light intensity through a tissue site based at least in part on the attenuated light received by the second detector; and estimating a concentration of an analyte of interest at the tissue based at least in part on the incident light (for example, an light intensity of the light source) detected by the first detector and the light intensity through the tissue site detected by the second detector. The second detector can include a set of detectors.

For purposes of summarizing the disclosure, certain aspects, advantages and novel features of the inventions have been described herein. It is to be understood that not necessarily all such advantages can be achieved in accordance with any particular embodiment of the inventions disclosed herein. Thus, the inventions disclosed herein can be embodied or carried out in a manner that achieves or optimizes one advantage or group of advantages as taught herein without necessarily achieving other advantages as can be taught or suggested herein.

### BRIEF DESCRIPTION OF THE DRAWINGS

Throughout the drawings, reference numbers can be re-used to indicate correspondence between referenced elements. The drawings are provided to illustrate embodiments of the inventions described herein and not to limit the scope thereof.

FIGS. 1A and 1B illustrate block diagram of example optical measurement devices.

FIG. 2 illustrates a flow diagram of example pulse oximetry device and/or patient monitoring device.

FIG. 3A illustrates a side view of an example detector.

FIG. 3B illustrates a top/bottom view of an example detector.

FIG. 4 illustrates examples of light absorption data collected by a detector having varying thickness.

### DETAILED DESCRIPTION

Beer's Law (also known as the Beer-Lambert Law) relates the attenuation of light to properties of a material. In particular, Beer's law states that absorbance of a material is proportional to the concentrations of the attenuating species in the material sample. The relationship between these parameters can be expressed as:

A=ϵ*b*c (1)

where A is the absorbance of the material at a given wavelength of light, ε is the molar absorptivity or extinction coefficient (L mol⁻¹ cm⁻¹), unique to each molecule and varying with wavelength, b is the length of the light path through the material (cm), and c is the concentration of an analyte of interest (mol L⁻¹).

In addition to absorbance, the concentration of an analyte of interest can be determined based on an intensity of the light transmitted through the material (I) and an intensity of incident light (I₀). The relationship between these parameters can be expressed as:

I=I₀*e^{-ϵ*b*c} (2)

Accordingly, the concentration c of an analyte of interest can be determined based on a known intensity of light transmitted through the solution I, an intensity of the incident light I₀, a pathlength b, and the molar absorptivity ϵ at a particular wavelength λ.

However, generally, a precise, real-time measurement of intensity of the incident light (I₀) is not known. Conventionally, I₀ has been measured in a variety of ways which either do not produce accurate measurements or produce measurements with degrading accuracy over time.

For instance, I₀ can be measured accurately during manufacturing. However, because the light source will experience real-time fluctuations and power degradation over time, using the manufacturing I₀ measurement will eventually lead to inaccuracies and miscalculations of physiological parameters such as glucose.

Moreover, I₀ can also be determined by sampling a portion of the projected light. However, the sampled portion of light may not accurately represent the entire beam of projected light unless the light is properly mixed. Mixing light properly usually requires an integrating sphere coated with a diffuse reflective material such as a fluoropolymer. Further, accurate light mixing requires a larger diameter sphere which is in opposition to miniaturize a sensor as much as possible.

In other examples, a reference photodiode can be utilized underneath a side of a light source to capture light emitted out the back of a chip. However, this technique does not provide an accurate representation of the light emitted out the front spectrally and it becomes difficult to capture more than one light source on a single reference photodiode if there are multiple light sources in a sensor.

The forgoing difficulties exemplify a need for an improved I₀ measuring technique that provides accurate, real-time I₀ detection with low quantum efficacy (for example, low light absorption) and takes up limited space. By accurately determining I₀, a system can determine various analyte concentrations or changes in concentration in various kinds of biological (for example, living) or non-biological (for example, non-living) material. In some cases, based at least in part on the concentration of an analyte of interest, a system can determine various other predictions or determinations. For example, in pulse oximeter or spectrographic systems, based on the concentration of an analyte of interest, the system can determine or predict physiological parameters, such as glucose or other analyte values. Similar predictions or determinations can be made when the measuring site includes non-biological material.

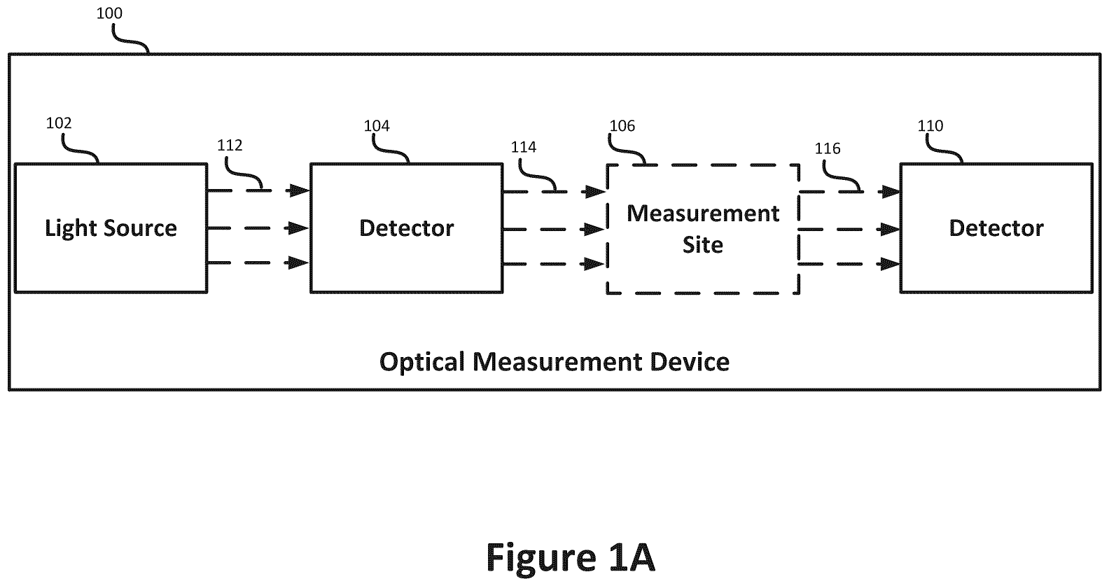

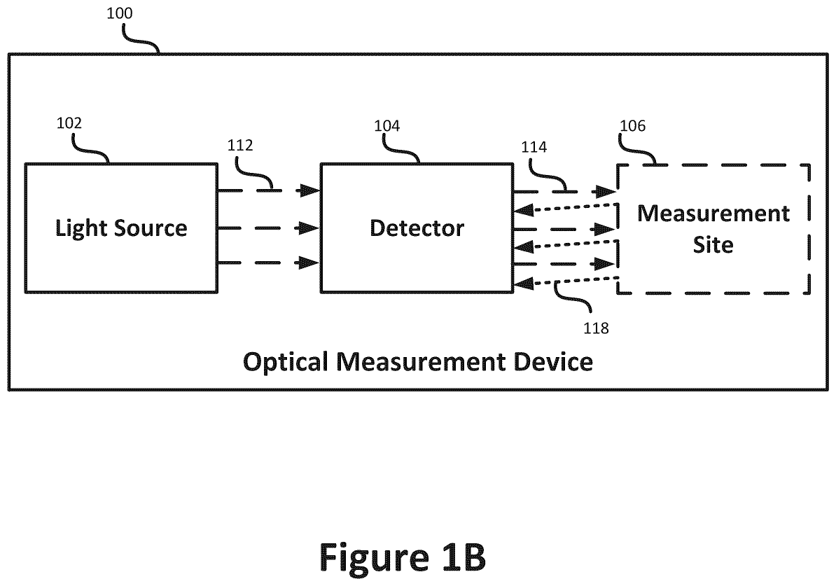

FIG. 1A illustrates block diagram of an example optical measurement device. The optical measurement device 100 includes a light source 102 configured to emit light towards a measurement site 106, a first detector 104 configured to detect incident light 112 of the light source 102, and a second detector 110 configured to detect light 116 transmitted through the measurement site 106. As illustrated, the first detector 104 is proximal the light source 102 and distal the second detector 110 with respect to the measurement site 106. In addition, the second detector 110 is distal the light source 102 with respect to the first detector 104 and with respect to the measurement site 106. In some cases, the optical measurement device 100 is a pulse oximetry device or a spectrophotometer and the measurement site 106 is a tissue site of a patient. However, the optical measurement device 100 can be any optical measuring device and the measurement site can include any biological or non-biological material.

The light source 102 can include one or more light emitting diodes (LEDs), superluminescent LEDs (SLEDs), lasers, etc. for transmitting optical radiation (for example, light at one or more wavelengths) 112 into or reflecting off the measurement site 106. For case of reference, the light (for example, the arrows) depicted in FIG. 1A is characterized at multiple stages within the optical measurement device 100. However, in the example illustrated in FIG. 1A, only the light source 102 emits light. The numbered distinctions (112, 114, 116) represent the differing intensities and wavelengths of light from the light source 102 as the light travels from the light source 102 to the second detector 110. For instance, the first set of arrows 112 represents incident light projected from the light source 102 and detected by the first detector 104; the second set of arrows 114 represents light as it emerges from the first detector 104 and reaches the measurement site 106; and the third set of arrows 116 represent light as it emerges from the measurement site 106. Accordingly, each stage of light 112, 114, 116 may have a differing intensity due to, for instance, absorption and/or attenuation.

The first detector 102 is positioned proximal to the light source 102 so as to advantageously detect incident light 112 or an intensity of incident light, I₀, as the incident light 112 emerges from the light source 102. This positioning between the light source 102 and first detector 102 allows for a highly accurate real-time I₀ detection by the first detector 104 before any significant reduction in intensity of the light projected by the light source 102.

At least a portion of the first detector 104 can be translucent or partially transparent, thereby acting as a window (and a photodiode) from which all light projected from the light source 102 will pass through prior to reaching the measurement site 106. Advantageously, the photodiode (for example, the translucent portion of the first detector 104) receives all (or a substantial portion) of the incident light 112 projected by the light source 102 and can output a signal responsive to the detected light. Thus, unlike the light sampling method mentioned above, the detected light is accurate representation of the light coming from the light source 102. Additionally, the first detector 104 can be positioned such that it detects little or no backscatter light.

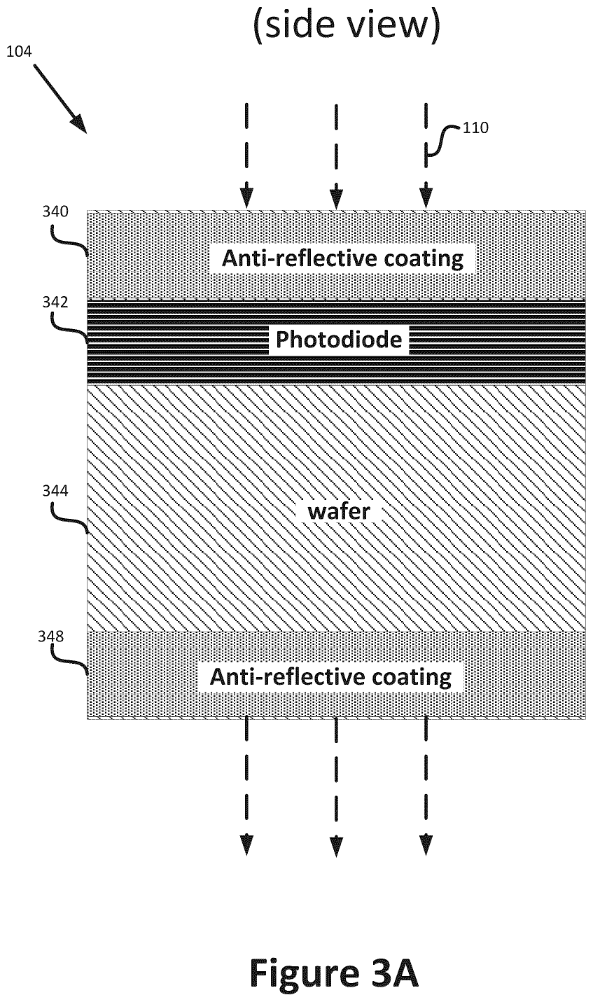

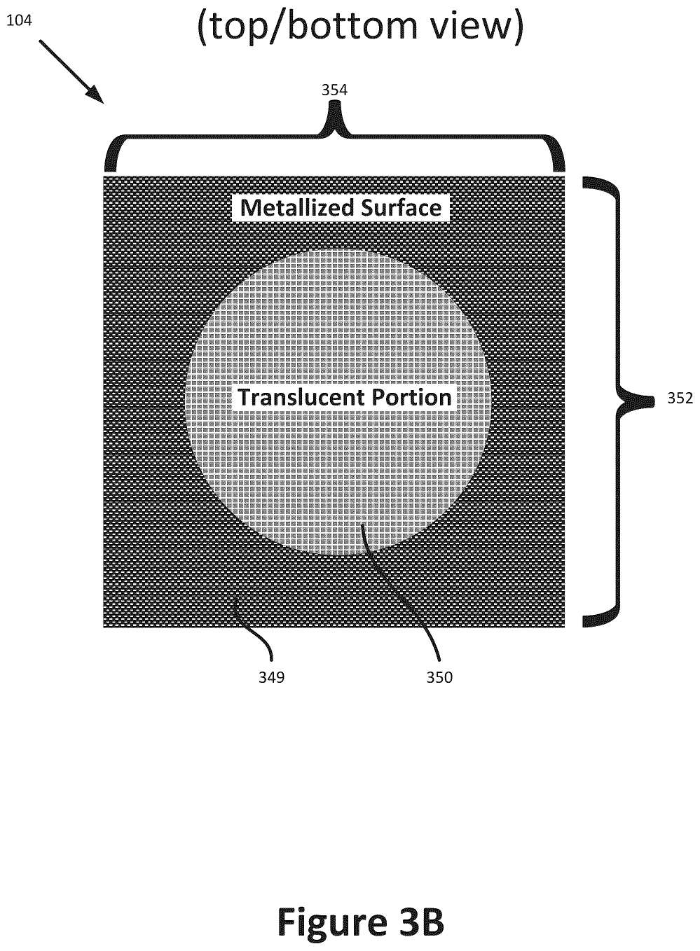

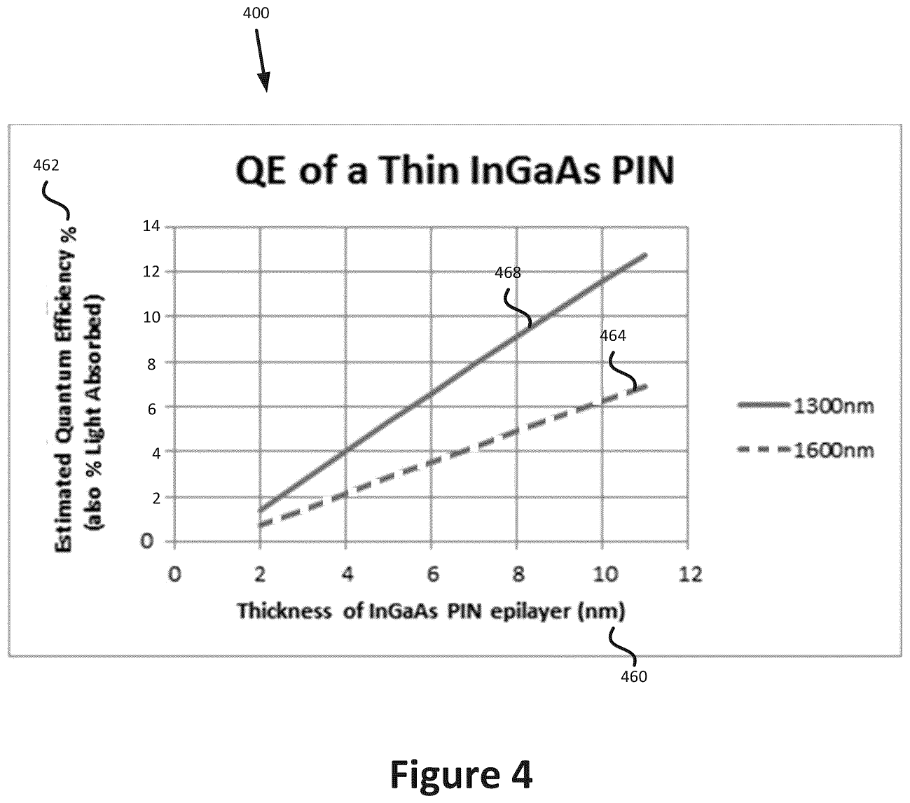

Additionally, the first detector 104 can include other advantageous properties (as described in more detail with respect to FIGS. 3-4) that reduce an amount of incident light 112 absorbed by the first detector 104 during detection. Accordingly, the low quantum efficiency (for example, low light absorption) of first detector 104 indicates that the light 114 transmitted to the tissue site 106 is substantially equal to the incident light 112 from the light source 102. For instance, the majority of light (for example, about 90%, 92%, 94%, 96%, or 98% (+/−1%)) of the incident light 112 can reach the measurement site 106 despite first passing through the first detector 104. In other words, the first detector 104 advantageously absorbs a small percentage of the incident light 112. For example, the first detector 104 can absorb less than 10%, less than 5%, or less than 2% (+/−a few %) of the incident light 112.

By permitting the majority of the light 112 to pass through, the first detector 104 is able to generate a signal corresponds to the intensity of incident light 112 without greatly affecting the intensity of light 114 transmitted to the measurement site 106. Accordingly, the incident light 112 and the light 114 emerging from the first detector 104 have substantially similar intensities. Furthermore, in some cases, the small reduction in light intensity caused by the first detector 104 is negligible because the source power is much higher than after the light has traveled through the measurement site 106.

The second detector 110 can include one or more detectors such as a set of multi-detectors. The second detector 110 is distal the light source 102 and proximal the measurement site 106 with respect to the first detector 104. The second detector 110 is positioned to detect the light 116 (or intensity of light 116) transmitted through the material the light 116 as it emerges from the measurement site 106. After attenuation by the measurement site 106, the second detector 110 detects the attenuated light 116 and outputs a signal responsive to the detected attenuated light 116. In some examples, the second detector 110 includes one or more photodiodes that generate one or more currents proportional to the intensity of the detected light 116.

In some cases where the optical measurement device 100 is a pulse oximetry device attached to a patient's finger, the second detector 110 can be positioned at the patient's fingertip opposite the fingernail so as to detect the light 116 as it emerges from the finger tissue site. The pulse oximetry device can include a clothespin-shaped housing (not shown) having a contoured bed conforming generally to the shape of a finger. For instance, the pulse oximetry device can include an enclosure for receiving a patient's finger. The enclosure can be formed by an upper section pivotably connected with a lower section. The upper section can include the light source 102 and the first detector 104, and the lower section can include the second detector 110. The upper section can be biased with the lower section to close together around a pivot point and thereby sandwiching the measurement site 106 (for example, the finger tissue site).

FIG. 1B illustrates block diagram of another example of optical measurement device 100. Similar to FIG. 1A, in this example, the optical measurement device 100 includes a light source 102 and a detector 104. However, in this example, the detector 104 is utilized to detect light transmitted by the light source and/or light reflected or refracted 118 by the measurement site 106. As illustrated, the optical measurement device 100 is configured to be positioned such that the light source 102 is proximal the detector 104 with respect to the tissue site 106.

As described herein, at least a portion of detector 104 can be translucent or partially transparent, thereby acting as a window (and a photodiode) from which all light projected from the light source 102 will pass through prior to reaching the measurement site 106 of the patient. Similarly, the detector 104 can be positioned proximate the measurement site 106 such that it receives light 118 reflected and/or refracted from the measurement site. Thus, the detector 104 can detect a light intensity of the light source 102 and/or light intensity of reflected and/or refracted light 118, and the detector 104 can output one or more signals responsive to the light detections.

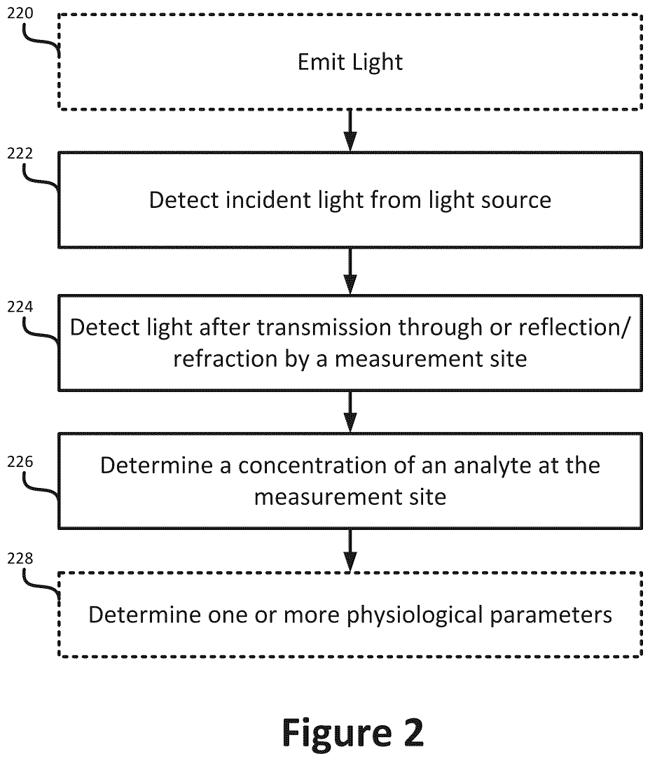

FIG. 2 illustrates an example flow diagram for determining a concentration of an analyte of the measurement site using the optical measurement device 100 of FIG. 1A or 1B. At block 220, the light source 102 transmits, projects, or emits light 112 towards the measurement site 106. As described herein, the measurement site 106 can include one or more various biological or non-biological material. For example, the measurement site can be a tissue site of a patient. Further, as described above with respect to FIG. 1A or 1B, the light source 102 can include one or more LEDs or SLEDs for transmitting optical radiation. In some cases, the light source 102 can emit light having multiple wavelengths such as red, infrared (IR), near IR, or the like.

At block 222, the detector 104 detects incident light 112 emitted by the light source 102. As described above with respect to FIG. 1A or 1B, at least one portion of the detector 104 can be translucent. The detector 104 is positioned proximal to the light source 102 with respect to the measurement site 106, and can act as a window from which all, or substantially all, light projected from the light source 102 will pass through prior to reaching the measurement site 106. Additionally, the detector 104 can advantageously have low quantum efficiency, thereby ensuring the light 114 transmitted to the measurement site 106 has substantially the same intensity as the light projected 112 by the light source 102. The detector 104 can include a photodiode that generates a current proportional to the intensity of the incident light 112. Accordingly, using the detector 104, a system advantageously can accurately determine the real-time I₀ without substantially reducing the intensity of light transmitted to the measurement site 106.

At block 224, a detector can detect light 116 transmitted through the measurement site, after attenuation by the measurement site 106. In addition or alternatively, a detector can detect light 118 transmitted reflected or refracted from the measurement site 106. For example, as illustrated in FIG. 1A, the detector 110 can detect light 116 as it emerges from the measurement site 106. As another example, as illustrated in FIG. 1B, the detector 104 can detect light 118 as it reflects or refracts from the measurement site 106. The detector 104 or 110 detects the light 116 or 118 and outputs a signal responsive to the detected light 116 or 118. For example, the detector 104, 110 can be a photodiode that generates a current proportional to the intensity of the detected light 116, 118.

At block 226, a concentration of an analyte of interest of the measurement site 106 can be determined (for example, by one or more processors). For example, using the relationship of Equation 2, the concentration can be determined based at least in part on the detected incident light, the detected light transmitted through the measurement site, or the detect light reflected or refracted by the measurement site. Furthermore, in some cases, a transmittance of light, an absorbance of light, and/or a reflectance of light can be determined.

At block 228, the system determine one or more parameters based at least in part on the analyte concentration, transmittance of light, absorbance of light, or reflectance of light determined at block 226. For example, when the optical measurement device is a pulse oximeter or a spectrophotometer, the system can utilize a concentration of an analyte of interest, absorbance, transmittance, reflectance, or other data at a tissue site to determine one or more physiological parameters corresponding to a patient. For example, the system can determine or predict a measurement indicative of a blood constituent of interest, such as glucose, oxygen saturation, methemoglobin, carboxyhemoglobin, glycated hemoglobin, respiration rate, pulse rate, total hemoglobin, other physiological parameters, or other data or combinations of data useful in determining a state or trend of wellness of a patient. An inverse model of the collected data at different blood glucose values can be created and used to predict glucose (or other analyte) values based at least in part on the measured tissue absorbance. Some other examples of parameters that can be used in the development of an inverse model include but are not limited to various measured temperatures (LED, tissue, ambient, photodiode, etc.) and absorbance of various reference materials measured real-time.

In some examples, a data collection system (not shown) can be provided which includes a signal processor, a user interface connected to the signal processor, a storage device and a network interface device, which are connected to the signal processor. The data collection system can include a user interface, such as a display. The data collection system can also include optional outputs alone or in combination with the display, such as a storage device and a network interface. The signal processor can include processing logic that determines measurements for desired analytes, such as glucose, based on the signals received from the one or more detectors 104, 110. The signal processor can be implemented using one or more microprocessors or subprocessors (for example, cores), digital signal processors, application specific integrated circuits (ASICs), field programmable gate arrays (FPGAs), combinations of the same, and the like.

FIG. 3A illustrates a side view of an example detector 104 of the pulse oximetry device 100 of FIG. 1A. In this example, the detector 104 includes four layers. In other examples, the detector 104 can have fewer or more than four layers. The layers can include the same, additional, or equivalent materials to the layers shown in FIG. 3A.

Here, the top layer 340 and the bottom layer 348 include an anti-reflective and/or anti-glare coating. This coating advantageously improves (for example, lowers) the quantum efficiency of the detector 104 (for example, the percentage of light absorbed by the detector 104) by virtually eliminating reflections of the detector 104. As described above with respect to FIG. 1A, it is desirable for the translucent portion of the detector 104 to pass (and not absorb) the light 112 projected from the light source 102. The less light absorbed by the detector 104, the more accurate the light intensity detected by the detector 104.

The second layer 342 includes a photodiode that converts the light 112 into current. In some examples, at least a portion of the second or photodiode layer 342 can be translucent. The photodiode layer 342 can have varying thicknesses across multiple embodiments. For example, the photodiode layer 342 can be as thick as 1, 2, 3, 4, 5, 6, 7, 8, 9, 10, 11, 12, 13, 14, or 15 nanometers (+/−a few nanometers) or can be as thick as a few micrometers. As mentioned below with respect to FIG. 4, the thickness of the photodiode layer 344 can affect the quantum efficiency or light absorption of the detector 104. Thus, in some examples, the photodiode layer 342 can advantageously be thin to provide improved (for example lowered) quantum efficiency. The photodiode layer 342 can include a combination of one or more of an Indium-Gallium-Arsenide (InGaAs) PIN photodiode or a silicon (Si) PIN photodiode. For example, the photodiode 342 may include a layer of Silicon PIN photodiode and an over-layer of Indium-Gallium-Arsenide (InGaAs) PIN photodiode.

The third layer 344 includes a wafer or thin slice of semiconductor material. In some examples, the third layer 344 includes an N-type Indium Phosphide (N—InP) wafer. The third layer 344 can have one of a plurality of thicknesses. For instance, the third layer 344 can be 1, 2, 3, 4, 5, 6, 7, 8, 9, 10, 11, 12, 13, 14, or 15, 20, 50, 100 micrometers thick (+/−a few micrometers).

FIG. 3B illustrates a top/bottom view of an example detector. Here, the detector 104 has a translucent portion 350 and an opaque portion 349. In some examples, the detector 104 can be fully or mostly translucent. While the translucent portion 350 is depicted as having a circular shape, the translucent portion 350 can take any shape. The detector 104 can include more than one translucent portion.

The opaque portion 349 can include a metalized surface. For example, the opaque portion 349 can include a metalized surface for anode/cathode bond pads. As mentioned above, the with respect to FIG. 3A, the surface of the detector 104 can have an antireflective coating to improve quantum efficiency.

In some examples, the translucent portion can be generally circular and have a diameter of approximately 2, 4, 5, 6, 7, 8 mm (+/−a few millimeters). In some examples the area of the translucent portion can be approximately 5, 6, 7, 10, 20, 40 or 80 square millimeters (+/−a few square millimeters).

The detector 104 can take the shape of a rectangular prism, cube, cylinder, or any other shape. In some examples, the width 354 and/or length 352 of the detector 104 is approximately 3 millimeters. In other examples, the width 354 and/or length 352 can be in the range of 1 to 10 millimeters (+/−a few millimeters).

FIG. 4 illustrates examples of quantum efficiency data of a plurality of detectors 104 having photodiode layers of varying thicknesses. As described above, the projected light 112 from the light source 102 is transmitted through a photodiode layer 342 of the detector 104 prior to reaching the tissue site 106 of the patient. FIG. 4 illustrates how the thickness of the photodiode layer 342 (in this example an InGaAs PIN epilayer) affects the quantum efficiency (and total light absorbed) of the detector 104.

In FIG. 4, the X axis 460 of the line chart 400 indicates the thickness (in nanometers) of the InGaAs PIN epilayer of the detector 104. The Y axis 462 of the line chart 400 indicates the estimated quantum efficiency (also light absorbed) expressed as a percentage. The “quantum efficiency” is the ratio of light absorbed to light received (for example, light projected by the light source). Thus, if all photons of a certain wavelength are absorbed, then the quantum efficiency at that particular wavelength is unity. In other words, low light absorption corresponds to low quantum efficiency.

Line 464 represents data values for projected light with wavelength of 1600 nanometers and line 468 represents data values for projected light with wavelength of 1300 nanometers. As indicated by the positive slopes of the lines 464 and 468, as the thickness of the photodiode layer increases, the total light absorbed by the detector increases. Accordingly, in some examples, the detector 104 can have a thin photodiode layer (for instance, 2 or 3 nm) to advantageously reduce the amount of light absorbed by the detector 104, thereby providing the tissue site 106 with light having intensity similar to that of the light as it emerges from the light source 102.

In addition, lines 464 and 468 of FIG. 4 indicate that as the wavelength of projected light increases, the quantum efficiency decreases. Accordingly, in some examples, the light source emits light with longer wavelength (for instance, approximately 1600 nanometers) to advantageously reduce the amount of light absorbed by the detector 104.

Embodiments have been described in connection with the accompanying drawings. However, it should be understood that the figures may not be drawn to scale. Distances, angles, etc. are merely illustrative and do not necessarily bear an exact relationship to actual dimensions and layout of the devices illustrated. In addition, the foregoing embodiments have been described at a level of detail to allow one of ordinary skill in the art to make and use the devices, systems, methods, etc. described herein. A wide variety of variation is possible. Components, elements, and/or steps can be altered, added, removed, or rearranged. While certain embodiments have been explicitly described, other embodiments will become apparent to those of ordinary skill in the art based on this disclosure.

Conditional language used herein, such as, among others, “can,” “could,” “might,” “may.” “for example.” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the inventions described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope.

1. (canceled)

2. A method of determining a physiological parameter using an optical measurement device, the method comprising:
causing a light source to emit light, wherein a first detector positioned along a light path between the light source and a measurement site detects incident light emitted by the light source as it emerges from the light source, wherein at least some light emitted by the light source passes through a portion of the first detector prior to reaching the measurement site;
detecting, using a second detector, attenuated light, the attenuated light having been attenuated by the measurement site;
receiving, using one or more hardware processors, a first signal generated by the first detector, the first signal corresponding to the detected incident light;
receiving, using the one or more hardware processors, a second signal generated by the second detector, the second signal corresponding to the detected attenuated light; and
determining, using the one or more hardware processor, a physiological parameter based at least in part on the first signal and second signal.

3. The method of claim 2, wherein a region of the first detector that detects the incident light is different from the portion of the first detector through which light passes to reach the measurement site.

4. The method of claim 2, wherein the first signal is indicative of an intensity of the incident light.

5. The method of claim 2, the method further comprising determining an intensity of the incident light based at least in part on the first signal.

6. The method of claim 2, wherein the physiological parameter corresponds to a blood glucose concentration.

7. The method of claim 2, wherein the first detector is proximal to the light source with respect to the measurement site, wherein the measurement site comprises non-biological material.

8. The method of claim 2, wherein the first detector is proximal to the light source with respect to the measurement site, wherein the measurement site comprises biological material.

9. A detector for measuring incident light emitted by a light source, the detector comprising:
a first anti-reflective coating;
a second anti-reflective coating; and
a photodiode positioned between the first anti-reflective coating and second anti-reflective coating, wherein at least a portion of the photodiode is translucent.

10. The detector of claim 9, wherein the photodiode is 1-15 nm thick.

11. The detector of claim 9, comprising a detecting region configured to detect incident light.

12. The detector of claim 11, comprising a translucent region configured to allow passage of incident light through the detector.

13. The detector of claim 12, wherein the detecting region is different from the translucent region.

14. The detector of claim 12, wherein the detecting region overlaps with the translucent region.

15. The detector of claim 9, further comprising a wafer positioned between the photodiode and the second anti-reflective coating.

16. The detector of claim 15, wherein the wafer is 1-10 μm thick.

17. The detector of claim 15, wherein the wafer is a N-type Indium Phosphide (N—InP) wafer.

18. An optical measurement device comprising a light source and the detector of claim 9 configured to detect incident light emitted from the light source, wherein the detector is positioned in a light path of the light source.

19. The optical measurement device of claim 18, wherein the optical measurement device is configured to measure a physiological parameter, and the detector configured to measure incident light emitted from the light source prior to reaching a measurement site of a patient.

20. The optical measurement device of claim 19, wherein the optical measurement device comprising a second detector configured to detect attenuated light, the attenuated light having been attenuated by the measurement site.

21. The optical measurement device of claim 19, wherein the detector is configured to detect attenuated light, the attenuated light having been attenuated by the measurement site.
